(19) United States
(12) Patent Application Publication
(10) Pub. No.: US 2008/0275437 A1
(43) Pub. Date: Nov. 6, 2008

(54) CONNECTOR DEVICE FOR ELECTROPHYSIOLOGY PROBE

(76) Inventors: Albert K. CHIN, Palo Alto, CA (US); Alfredo R. CANTU, Pleasanton, CA (US); Peter CALLAS, Casto Valley, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/053,103

(22) Filed: Mar. 21, 2008

Related U.S. Application Data
(60) Provisional application No. 60/896,441, filed on Mar. 22, 2007.

(51) Int. Cl. A61B 18/18

(52) U.S. Cl. 606/33; 606/41

(57) ABSTRACT

A flexible connector for securing an ablation probe in a looped configuration around the pulmonary veins of a heart includes a flexible base member having first and second apertures dimensioned to receive the ablation probe and wherein the flexible base member is slidable along a length of the ablation probe when the ablation probe is positioned within the first and second apertures. The flexible base member further includes a third aperture dimensioned to receive a looped portion of the ablation probe. The flexible connector is advanced along the length of the ablation probe to form a crossover point with the returning portion of the ablation probe. A tether is secured to the flexible base member and is used to withdraw or remove the flexible connector.

This application claims priority to Provisional Application Ser. No. 60/896,441 entitled “Connector Device For Electrophysiology Probe” filed Mar. 22, 2007, which is incorporated herein by reference.

### FIELD OF THE INVENTION

The field of the invention generally relates to devices and methods for providing therapy to a patient, and more particularly to devices and methods for ablating heart tissue of the patient.

### BACKGROUND OF THE INVENTION

In electrophysiological therapy, tissue ablation is used to treat cardiac rhythm disturbances in order to restore the normal function of the heart. Normal sinus rhythm of the heart begins with the sinoatrial node (or “SA node”) generating a depolarization wave front that propagates uniformly across the myocardial tissue of the right and left atria to the atrioventricular node (or “AV node”). This propagation causes the atria to contract in an organized manner to transport blood from the atria to the ventricles. The AV node regulates the propagation delay to the atrioventricular bundle (or “HIS” bundle), after which the depolarization wave front propagates uniformly across the myocardial tissue of the right and left ventricles, causing the ventricles to contract in an organized manner to transport blood out of the heart. This conduction system results in an organized sequence of myocardial contraction leading to a normal heartbeat.

Sometimes, however, aberrant conductive pathways develop in heart tissue, which disrupt the normal path of depolarization events. For example, anatomical obstacles in the atria or ventricles can disrupt the normal propagation of electrical impulses. These anatomical obstacles can cause the depolarization wave front to degenerate into several circular wavelets that circulate about the obstacles. These wavelets, called “reentry circuits,” disrupt the normal activation of the atria or ventricles. As a further example, localized regions of ischemic myocardial tissue may propagate depolarization events slower than normal myocardial tissue. The ischemic region, also called a “slow conduction zone,” creates errant, circular propagation patterns, called “circus motion.” The circus motion also disrupts the normal depolarization patterns, thereby disrupting the normal contraction of heart tissue. The aberrant conductive pathways create abnormal, irregular, and sometimes life-threatening heart rhythms, called arrhythmias. An arrhythmia can take place in the atria, for example, as in atrial tachycardia (AT), atrial fibrillation (AFIB), or atrial flutter (AF). The arrhythmia can also take place in the ventricle, for example, as in ventricular tachycardia (VT).

Once the location of the sources of the aberrant pathways (called substrates) are identified, the tissue in the substrates can be destroyed, or ablated, by heat, chemicals, or other means of creating a lesion in the tissue, or otherwise can be electrically isolated from the normal heart circuit. One surgical approach for treating atrial arrhythmia, known generally as the MAZE III procedure, effectively creates an electrical maze in the atrium and precludes the ability of the atria to fibrillate. In this procedure, strategic incisions are performed to prevent atrial reentry circuits from forming and allow sinus impulses to activate the entire atrial myocardium, thereby preserving atrial transport function postoperatively. While the MAZE III procedure has proven effective in treating atrial arrhythmia, this operational procedure requires open-heart surgery and the introduction of substantial incisions within the interior chambers of the heart. Consequently, various other less invasive techniques have been developed to interrupt atrial fibrillation and restore normal sinus rhythm.

One such technique is the strategic ablation of heart tissue through the use of ablation catheters, which involves intravenously introducing catheters within the chambers of the heart and endocardially creating transmural ablation lesions within the myocardial tissue. For example, as part of the treatment for certain categories of atrial fibrillation, it may be desirable to create a curvilinear lesion around or within the ostia of the pulmonary veins (PVs), and a linear lesion connecting one or more of the PVs to the mitral valve.

Other, less invasive endoscopic techniques have been employed to create impulse-blocking scars using microwave ablation of atrial tissue. For example, the FLEX 10® microwave ablation system available from Boston Scientific/Guidant Corporation has been used to endoscopically treat atrial fibrillation. Generally, the procedure can be broadly divided into several steps. In the first step, multiple access ports are created on the right side of the chest. The pericardial sac is then opened parallel to the phrenic nerve to expose the right superior and inferior pulmonary veins. In the second step, the pericardial reflections investing the superior and inferior vena cavae are dissected to allow entry into the transverse and oblique sinuses, respectively. In the third step, guiding catheters are placed behind the cavae into the sinuses. In the fourth step, the ports created in the left chest and the left side of the pericardial sac is opened, revealing the guide catheters. In the fifth step, the catheters are retrieved, delivered outside the chest, tied together and then replaced so as to surround the pulmonary veins. In the sixth step, the catheter “loop” is used to draw the FLEX 10® device around the veins. Once the device's position is confirmed, a circumferential ablation line is laid down around the pulmonary veins on the left atrial wall. Particular details of the procedure are discussed in A. Saltman, Completely Endoscopic Microwave Ablation of Atrial Fibrillation on The Beating Heart Using Bilateral Thorascopy, CTSNet, www.ctsnet.org (Jul. 6, 2005).

Currently, surgeons that use the FLEX 10® ablation probe use endoscopic graspers in an attempt to close the “horseshoe” configuration of the FLEX 10® ablation probe wrapping around the pulmonary veins. In order to form a circumferential ablation pattern, the grasper grabs the flexible sheath which is pressed inferiorly toward its origin under the inferior vena cava. Closure of this gap may be monitored by visual inspection, typically via an endoscope. While holding the FLEX 10® ablation probe in place with the graspers, electrical energy is applied to ablate the heart tissue. Unfortunately, the current process requires the surgeon to hold and release the graspers during the ablation process which takes approximately two minutes for each segment of the FLEX 10® ablation probe. After one segment is complete, the surgeon releases the graspers and adjusts the FLEX 10® ablation element (e.g., microwave antenna) to the next segment and repeats the process for each segment. For ten such segments, there is a total of at least twenty minutes in which the surgeon must hold the graspers.

There thus is a need for a device and method that permits a surgeon performing ablation of cardiac tissue, such as a circumferential region around the pulmonary veins, without the need of graspers. Preferably, ablation could be performed without the need to hold portions of the microwave ablation probe during the ablation process. In this regard, the surgeon is not occupied during this time and may attend to other matters during the surgical procedure.

### SUMMARY

In one embodiment of the invention, a flexible connector for securing an ablation probe device in a looped configuration includes a flexible base member having first and second apertures dimensioned to receive the ablation probe and wherein the flexible base member is slidable along a length of the ablation probe when the ablation probe is positioned within the first and second apertures. The flexible base member further includes a third aperture dimensioned to receive a looped portion of the ablation probe. The flexible connector is advanced along the length of the ablation probe to form a crossover point with the returning portion of the ablation probe. A tether is secured to the flexible base member to withdraw or remove the flexible connector after the ablation process.

The flexible base member may comprise a substantially flat base when not loaded onto the ablation probe. The flexible base member may have any number of shapes including triangular, polygonal, circular, oval, and the like. The flexible base member may also include a tubular or cylindrical base having multiple apertures therein.

The tether may be secured to the flexible base member via a reinforcing member like an anchor disposed in the flexible base member. Alternatively, the tether may be secured to the flexible base member via one or more of the apertures. For example, the tether may be tied to itself after passing through one or more of the apertures. The tether may include a suture or biocompatible filament. In one embodiment, the flexible connector is deployed using an insertion tool that has a lumen for passage of the tether. The tubular member may be used to push or advance the flexible connector along a length of the ablation probe.

In another embodiment, an ablation probe includes an elongate member having a proximal end and a distal end, the elongate member including one or more ablation elements (e.g., microwave antenna) disposed at or near the distal end. The ablation probe further includes a connector for securing a portion of the elongate member in a looped configuration, the connector having a base portion with an aperture dimensioned such that the base portion is slidable along a length of the elongate member, the connector including a tab projecting from the base portion and including an aperture therein, the aperture of the tab being oriented substantially perpendicular to the aperture of the base portion. The connector may have an optional tether secured thereto for retrieving the connector after distal advancement to form the looped configuration. In addition, the probe may include an insertion tool that that is used to advance the connector along a portion of the length of the ablation probe.

In another aspect of the invention, a method of position an ablation probe around the pulmonary veins of a patient include providing a flexible connector device on a proximal portion of the ablation probe. The flexible connector device includes a tether attached thereto. The ablation probe is routed around the pulmonary veins and a distal end of the ablation probe is inserted in an aperture contained in the flexible connector device. The flexible connector device is then advanced along a length of the ablation probe to form a looped portion around the pulmonary veins. For example, an elongate pusher member or insertion tool may be used to advance the flexible connector device. Once the looped portion is formed around the pulmonary veins, electrical energy may then be applied the ablation probe to ablate cardiac tissue. During the ablation process, there is no need to hold together the ablation probe using graspers or the like. The flexible connector may be withdrawn in a proximal direction by pulling on the tether.

### BRIEF DESCRIPTION OF THE DRAWINGS

FIG. 1A is a perspective view of a flexible connector according to one embodiment.

FIG. 1B is a top down plan view of a flexible connector according to another embodiment.

FIG. 1C is a top down plan view of a flexible connector according to another embodiment.

FIG. 1D is a top down plan view of a flexible connector according to another embodiment.

FIG. 1E is a perspective view of a flexible connector according to another embodiment. The flexible connector is the form of a tube or cylinder.

FIG. 2A illustrates a top down plan view of an embodiment of a flexible connector that is integrated with an ablation probe.

FIG. 2B illustrates a cross-sectional view of the flexible connector taken along the line A-A′ in FIG. 2A.

FIG. 3A illustrates a flexible connector having a tether secured thereto according to another embodiment. Also illustrated is an insertion tool having a lumen therein dimensioned to receive the tether.

FIG. 3B illustrates a flexible connector having a tether secured thereto according to another embodiment. The tether is secured via multiple apertures.

FIG. 3C illustrates an insertion tool having a biased distal lumen.

FIG. 4 illustrates an ablation probe suitable for use with a flexible connector device.

FIG. 5 illustrates a flexible connector partially mounted to a portion of the ablation probe. Two of the three apertures are shown containing the probe while the third remaining aperture is open.

FIG. 6 illustrates a flexible connector partially mounted to a portion of the ablation probe according to another embodiment.

FIG. 7 illustrates a flexible connector positioned on the ablation probe at a crossover point around the pulmonary veins of the left atrium of a heart.

FIGS. 8A and 8B illustrate a method of using an ablation probe having a flexible connector according to one embodiment.

### DETAILED DESCRIPTION

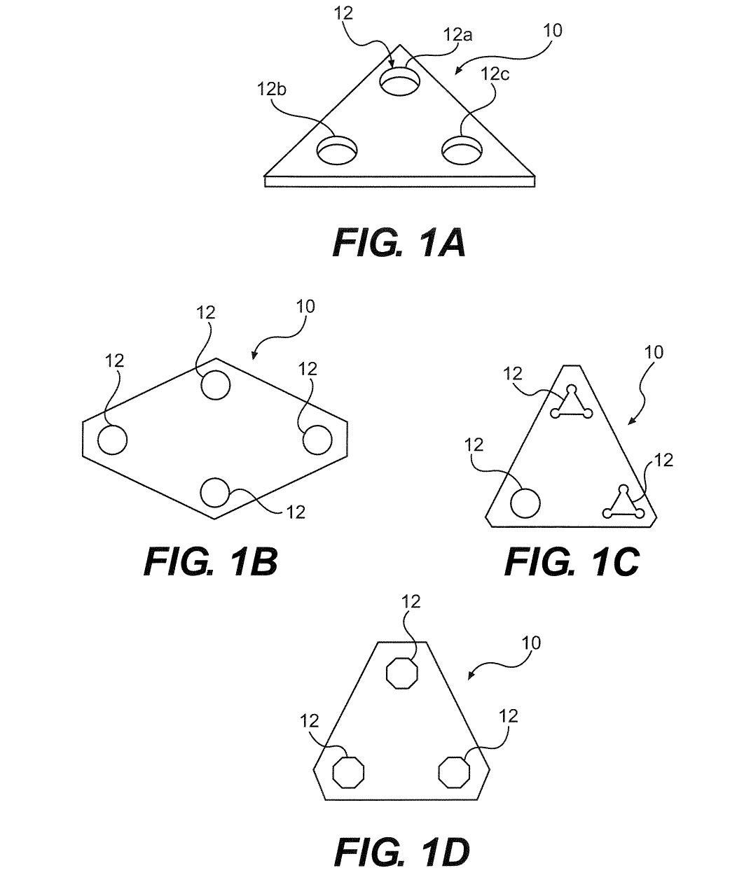

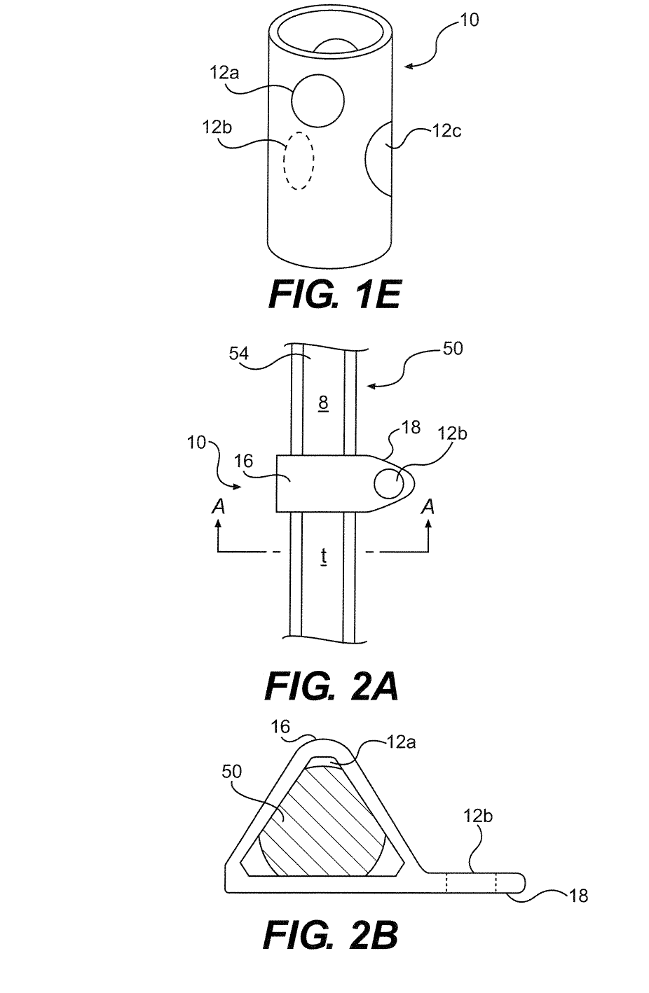

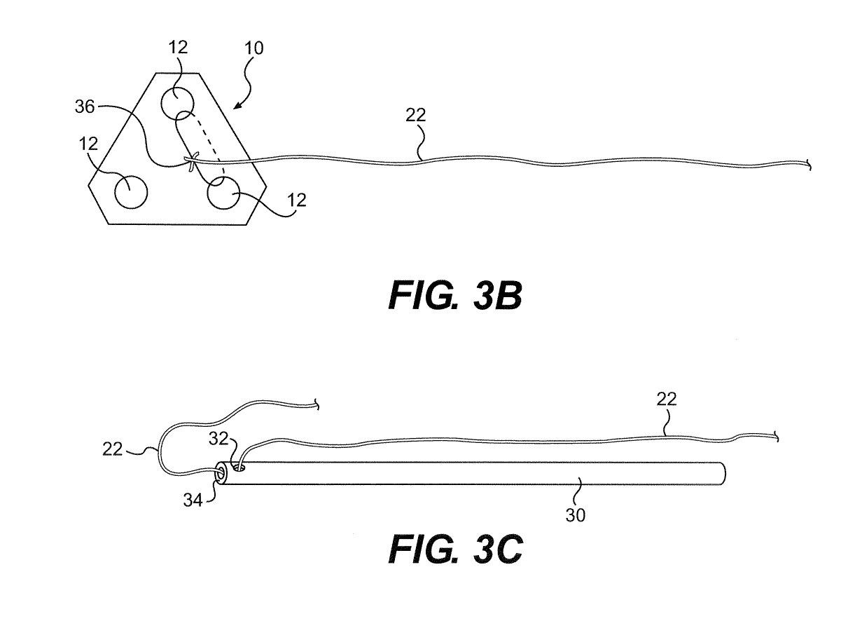

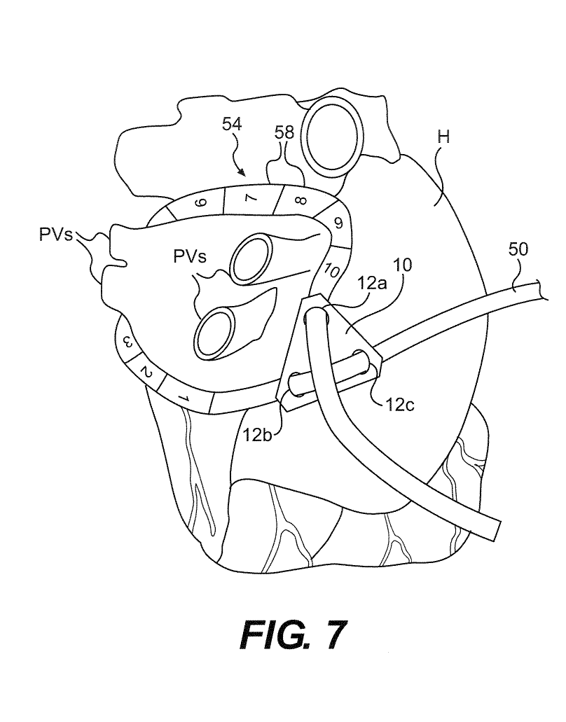

FIGS. 1A-1E, 2A, and 2B illustrates various embodiments of a flexible connector 10 that is used to adjust and secure the crossover point of an ablation probe 50 of the type illustrated in FIG. 4. FIG. 1A illustrates a flexible connector 10 in the shape of a triangle that includes a plurality of apertures 12. Three such apertures 12a, 12b, 12c are illustrated in the embodiment of FIG. 1A although additional apertures 12 may also be located within the flexible connector 10 (e.g., four apertures 12 in FIG. 1B). The flexible connector 10 may be made from a biocompatible, flexible material. For example, the flexible connector 10 may be formed from a flexible polymer or plastic-based material that has a degree of pliability such that, when positioned on the ablation probe 50, the flexible connector 50 is able to bend or flex to permit the formation of the intersecting crossover configuration as illustrated in FIG. 7. The flexible connector 10 may be molded, stamped, or cut to the desired shape and size. One or more portions of the flexible connector 10 may be radiopaque. For example, a radiopaque paint or coating could be applied to all or a portion of the flexible connector 10. Alternatively, the flexible connector 10 may be formed from a radiopaque material such as radiopaque silicon or use a radiopaque additive.

The apertures 12 contained within the flexible connector 10 are dimensioned such that the ablation probe 50 may be inserted therein. Preferably, the apertures are dimensioned such that the ablation probe 50 snugly fits within the respective apertures 12. In this regard, the flexible connector 10 may be slid along a portion of the ablation probe 50 while at the same, time, maintaining the crossover point when positioned into place. The apertures 12 may have any number of geometrical profiles. For example, the apertures 12 may be circular as illustrated in FIGS. 1A and 1B. Alternatively, the apertures 12 may be triangular such as those illustrated in FIG. 1C or polygonal as illustrated in FIG. 1D. In addition, the geometrical profiles may be different in a single flexible connector 10 as illustrated in FIG. 1C.

Similarly, the actual shape of the flexible connector 10 may vary. Typically, in one embodiment, when not loaded onto the ablation probe 50 the flexible connector 10 has a substantially flat profile. The flexible connector 10 may be formed in a polygonal shape such as those illustrated in FIGS. 1A-1D. For example, the flexible, connector 10 may be triangular or even rhombus-like. Of course, other geometrical shapes may also be used such as square, rectangular, circular, oval, and the like.

FIG. 1E illustrates an alternative embodiment of a flexible connector 10. In this embodiment, the flexible connector 10 is formed in the shape of a cylinder or tube. For example, a polymer or plastic-based tubing may have a number of apertures 12 formed therein. As seen in FIG. 1E, at least one aperture 12a is oriented generally perpendicular to the remaining two apertures 12b, 12c. In this regard, when the flexible connector 10 is loaded onto the ablation probe 50 a good crossover point for the intersecting segments is formed. The apertures 12 may be drilled or punched in a segment of tubing forming the flexible connector 10.

In one embodiment, the flexible connectors 10 may be sold or distributed separate from the ablation probe 50. In this embodiment, prior to use the flexible connector 10 is loaded onto the ablation probe 50. For example, the flexible connector 10 may be loaded onto the ablation probe 50 by inserting the distal end or tip 70 (shown in FIG. 4) of the ablation probe 50 into two of the apertures 12. If the ablation probe 50 is a FLEX 10® device, the filaments 72 secured to a guide lead 71 may also be fed through the two apertures 12. The flexible connector 10 may then be advanced along a length of the ablation probe 50 toward the proximal handle 52. The flexible connector 10 is advanced to place that is generally proximal with respect to the microwave ablation region 54 of the probe 50.

FIG. 2A illustrates one embodiment of a flexible connector 10 that is integrated onto the ablation probe 50. In this embodiment, the flexible connector 10 is pre-loaded onto the ablation probe 50. As illustrated in FIG. 2A, the flexible connector 10 is shown positioned between the “7” and “8” positions of the ablation region 54 of the probe 50. Typically, prior to placement, the flexible connector 10 is located proximal with respect to the ablation region 54 and then advanced distally once the probe 50 is in place. In the embodiment of FIG. 2A, the flexible connector 10 includes a base portion 16 that is slidable along a length of the probe 50. The base portion 16 may be made of a polymeric or plastic-type material as described herein. As seen in FIG. 2B, the base portion 16 includes an aperture 12a for receiving the elongate body 66 of the ablation probe 50. The ablation probe 50 is snugly disposed within the aperture 12a such that the base 16 may be slide in the proximal and distal directions as the base 16 is pushed or pulled relative to the probe 50. In one aspect, the base 16 is designed to be pushed and/or pulled directly by the hands of the operating surgeon. In still another aspect, however, a separate tool (not shown in FIG. 2A) may be used to advance and retract the base 16 along the length of the probe 50.

Referring to FIG. 2B, the flexible connector 10 includes a tab 18 projecting generally perpendicular to the base 16 (in the direction of aperture 12a). The tab 18 includes an aperture 12b therein that is dimensioned to receive the ablation probe 50. The aperture 12b is generally oriented perpendicular to the aperture 12a contained in the base 16. The aperture 12b is dimensioned such that the ablation probe 50 fits snugly therein. The tab 18 along with the base 16 is slidable along a length of the probe 50 such that the crossover point can be set and maintained. In this regard, the probe 50 is maintained in the looped configuration as illustrated in FIG. 7.

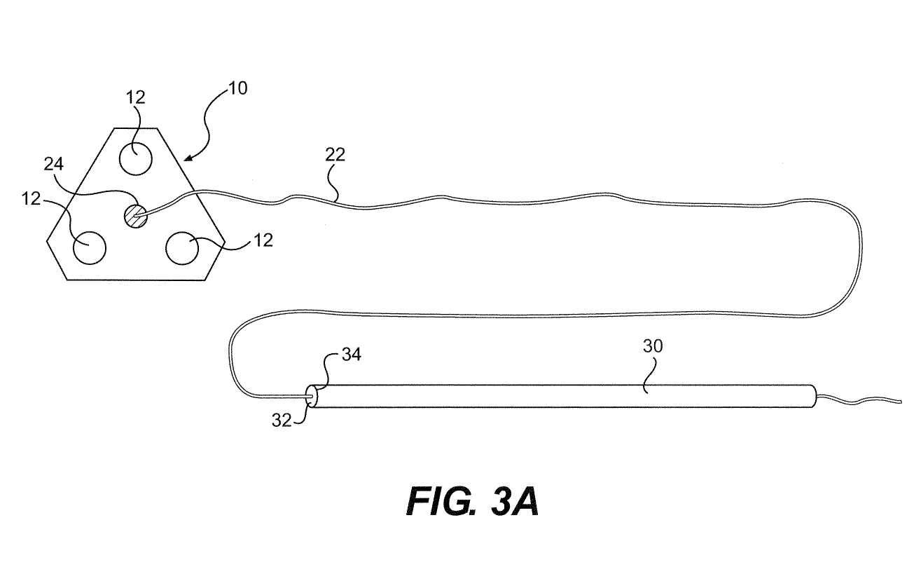

FIG. 3A illustrates another embodiment of a flexible connector 10. In this embodiment, a tether 22 is secured to the flexible connector 10 at one end while the other end is free. The tether 22 may comprise, for example, a surgical suture. The tether 22 may be secured to the flexible connector 10 via an anchor member 24 that is bonded to or formed integral with the flexible connector 10. As seen in FIG. 3A, the anchor member 24 is situated within the central portion of the flexible connector 10. For example, the tether 22 may be secured to an anchoring disc 24 of reinforcing material contained within the central portion of the flexible connector 10. The anchoring disc 24 may be made of a fabric, plastic, or metal that may be bonded to the flexible connector 10. For example, an adhesive or the like may be used to bond the anchoring disk 24 to the flexible connector 10. Alternatively, the anchoring disc 24 may be molded directly into the flexible connector 10. The purpose of the anchoring member 24 is to ensure that the tether 22 does not detach from the flexible connector 10 when tension is applied to the tether 22 to retract the flexible connector 10.

Still referring to FIG. 3A, an insertion tool 30 in the form an elongate pusher member having a lumen 32 therein is used in connection with the flexible connector 10. The lumen 32 of the insertion tool 30 is sized such that the tether 22 is slidably disposed therein. The insertion tool 30 may be made of a plastic, polymeric material, or metal, and have sufficient columnar strength such that the tool 30 does not buckle when a force is applied against the flexible connector 10. During use, the tether 22 is fed through the lumen 32 of the insertion tool 30 and a distal end 34 of the tool 30 abuts against the flexible connector 10. The length of the insertion tool 30 may be such that at least a portion extends external to the body cavity. The insertion tool 30 thus provides a way for the flexible connector 30 to be advanced endoscopically without the need to have the patient's chest cavity opened via, for instance, a sternotomy or large thoracotomy. The insertion tool 30 may alternatively contain a biased distal lumen 32 as seen in FIG. 3C for easier threading of the tether 22.

FIG. 3A illustrates an alternative embodiment in which the tether 22 is secured to the flexible connector 10 using the apertures 12. The tether 22 may be tied off using a knot 36 after passing through one or more of the apertures 12. While FIG. 3A shows the tether 22 passing through two apertures 12, it should be understood that the tether 22 may be secured via a single aperture 12 or, alternatively, more than two apertures 12. Also, a separate hole or passageway (not shown) may be formed in the flexible connector 10 that is used to secure the tether 22.

FIG. 4 illustrates an ablation probe 50 that may be used in connection with the flexible connectors 10 described herein. The ablation probe 50 of FIG. 4 is the FLEX 10® ablation device sold by Boston Scientific/Guidant Corporation. The ablation probe 50 includes a proximal handle 52 that is grasped by the surgeon during the ablation process. The handle 52 includes a sliding ring 56 that is used to move the microwave ablation antenna (not shown) proximally and distally within the probe 50 to apply microwave energy within the ablation region 54. The ablation region 54 contains a number of markers 58 that are used to mark the location where the ablation antenna is located and thus ablation will occur. The handle 52 includes corresponding position indicators 60 that indicate to the surgeon the location of where ablation is to take place. The location is adjusted by sliding movement of the ring 56 relative to the handle 52.

Still referring to FIG. 4, the ablation probe 50 includes a cable 61 (e.g., coaxial cable) that carries the electrical signal for microwave ablation. The cable 61 may then be connected to a microwave generator such as the Guidant 1000 Series Microwave Generator available from Boston Scientific/Guidant Corporation (not shown). The ablation probe 50 includes a metallic shaft portion 62 that terminates into a flexible sheath 64 that contains the moveable microwave antenna. The flexible sheath 64 terminates in a distal end 70. A flexible guide lead 71 is secured to the distal end 70 of the flexible sheath 64. In addition, one or more filaments 72 may be secured to the distal end of the guide lead 71 that can be used, for example, to secure the ablation probe 50 to a delivery or routing catheter. For example, the filaments 72 may be used to secure the ablation probe 50 to the proximal end of the routing catheter by proximally-located holes in the routing catheter. For example, the ablation probe 50 may be used in connection with the FLEX Guide routing tool sold by Boston Scientific/Guidant Corporation. Further details of the ablation probe 50, as well as alternative ablation probes that can be used in connection with the flexible connectors 10 are disclosed in U.S. Pat. Nos. 6,471,696 and 7,033,352, and U.S. Patent Application Publication No. 2003/0163128A1, which are expressly incorporated herein by reference.

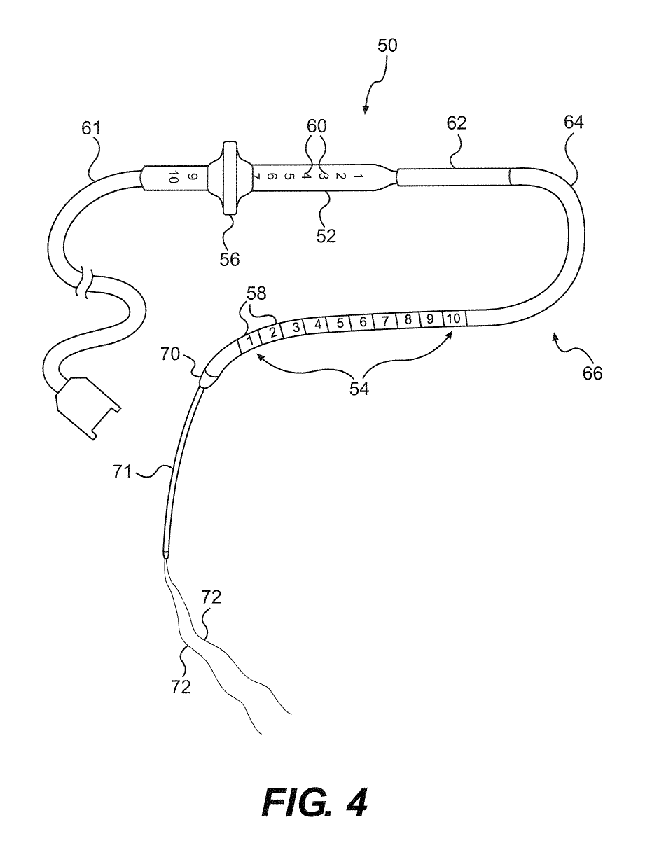

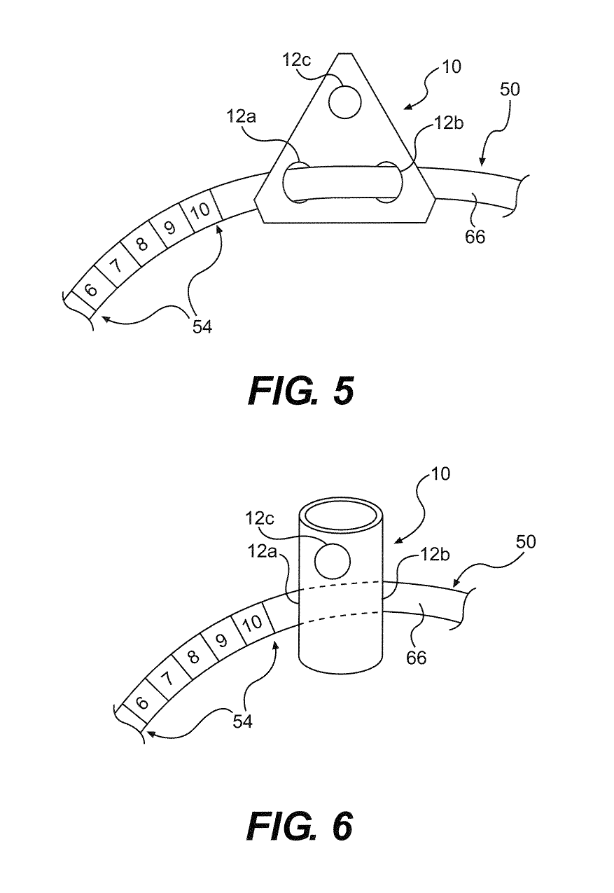

FIGS. 5 and 6 illustrate a flexible connector device 10 partially positioned on an ablation probe 50. The flexible connector device 10 is shown being loaded onto the elongate body 66 of the ablation probe 50 just proximal with respect to the ablation region 54. As seen in FIGS. 5 and 6, the ablation probe 50 is shown passing through two of the apertures 12a, 12b contained in the flexible connector 10. The remaining third aperture 12c remains open. The configuration of FIGS. 5 and 6 illustrates the location of the flexible connector 10 prior to placement of the ablation probe 50 around the pulmonary veins of the patient. In addition, it should be understood that the flexible connector 10 may be positioned further proximal with respect to the ablation region 54 than the position illustrated in FIGS. 5 and 6.

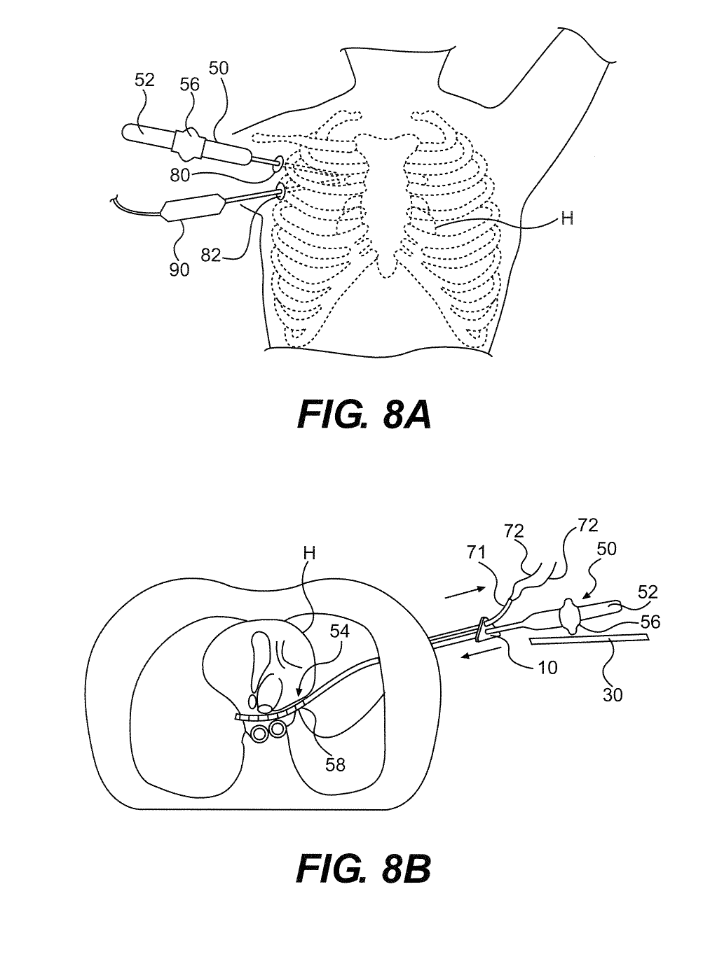

FIG. 7 illustrates the ablation probe 50 positioned about the pulmonary veins (PV) of a patient's heart. The ablation probe 50 is shown in the looped configuration with the flexible connector 10 advanced to form a crossover point adjacent to the pulmonary veins. The crossover point for a particular patient will vary with patient anatomy. The flexible connector 10 may be slid down or along a length of the elongate body 66 of the ablation probe 50 manually by the surgeon (e.g., in an open heart procedure) or, alternatively, the flexible connector 10 may be advanced along a length of the elongate body 66 by using an insertion tool 30 of the type illustrated in FIG. 3A. The insertion tool 30, for example, may be used when the ablation probe 50 is positioned endoscopically as illustrated in FIGS. 8A and 8B. The apertures 12 within the flexible connector 10 are dimensioned such that as the flexible connector 10 is advanced along the length of the elongate body 66 to a crossover point like that illustrated in FIG. 7, the flexible connector 10 does not move after placement. For example, if an insertion tool 30 is used to place the flexible connector 10, once the desired crossover point has been reached the insertion tool 30 may be removed, leaving the flexible connector 10 in place to hold the ablation probe 50 in a substantially stationary manner around the PVs.

FIGS. 8A and 8B illustrate one exemplary manner of placing the ablation probe 50 about the PVs of the heart (H) of a patient in an endoscopic manner. FIG. 8A illustrates an access port 80 formed a thoracic region of the patient. The access port 80 may be formed in the subxiphoid region or between the patient's ribs. The access port 80 may include a puncture, incision, or access port. For instance, the port 80 may include a small incision (e.g., about 2 cm) made in the subxiphoid region or on a side of the patient (e.g., right side, as in this case). As seen in FIG. 8A a second port 82 may be formed in the patient's chest. As seen in FIG. 8A, this secondary port 82 is formed between the patient's ribs. The secondary port 82 may be used to pass a visualization device 90 such as an endoscope within the patient's thoracic cavity to visualization placement of the ablation probe 50 around the PVs of the patient. Of course, the second port 82 may be located in other areas of the patient's thorax.

FIG. 8B illustrates a cross-sectional view of the patient illustrating the ablation probe 50 positioned about the PVs of a patient's heart (H). The ablation probe 50 is shown with the flexible connector 10 being disposed on the ablation probe 50 and located proximal with respect to the ablation region 54. The filaments 72 and guide lead 71 are shown passing through the other remaining aperture 12 of the flexible connector 10. During placement, the ablation probe 50 is routed around the PVs and the distal end of the probe 50 is retrieved out the same port (e.g., port 80) and inserted into the aperture 12 of the flexible connector 10. As explained herein, a guide or routing device such as the FLEX Guide routing tool may be used to route the ablation probe 50 around the PVs.

The flexible connector 10 can then be advanced along the length of the ablation probe 50 to the crossover point as illustrated in FIG. 7. For example, the filaments 72 may be inserted into the lumen 32 of the insertion tool 30 and the tool 30 can be used to push the flexible connector 10 through the port 80 and into the body cavity. The insertion tool 30 can then further advance the flexible connector 10 to the crossover point as illustrated in FIG. 7. After placement of the flexible connector 10, the insertion tool 30 may be removed from the patient's thoracic cavity leaving the filament(s) 72 protruding from the port 80.

With the flexible connector 10 in place, the surgeon can then begin the ablation process. Typically, the surgeon will ablate the cardiac tissue by first selecting an antenna position using the sliding ring 56 on the handle 52. The antenna is energized typically for between one and two minutes for each segment 54 (typically at less than 64 Watts). After a particular segment has been ablated, the surgeon can adjust the ablation location using the sliding ring 56. Unlike prior methods in which the surgeon was required to hold the ablation probe 50 in place using graspers, there is no need to hold the ablation probe 50 in position with graspers since the flexible connector 10 securely positions the ablation region 54 of the ablation probe 50 about the patient's PVs. After completion of the process, the surgeon pulls on the filaments 72 in the proximal direction to withdraw and remove the flexible connector 10 from the ablation probe 10.

The connector offers numerous advantages over the prior method of using graspers to hold the ablation probe 50. As explained above, because there is no need to handle a separate grasping tool, the surgeon's hand is able to adjust the position of the ablation antenna with the free hand. Moreover, because the surgeon's hand is free from the grasper, it is possible for the surgeon to perform other actions like, for example, taking down the internal mammary artery (IMA).

In addition, because the flexible connector 10 secures the ablation probe 50 to the patient's heart (H) during the entire ablation procedure there is little or no risk that the ablation region 54 will move with respect to the underlying heart tissue. In contrast, in the prior method, this was a potential risk given the fact that the operating surgeon needed to hold the grasper manually during the entire ablation process. In addition, for endoscopic approaches, the use of the tether 22 to withdraw the flexible connector 10 avoids potential puncturing issues associated with using an endoscopic grasper. Due to the tightness of the space around the heart and the need to coordinate visualization of the endoscope 90 with manipulation of the endoscopic grasper may pose a risk of puncturing the heart tissue through errant grasper movements. The tether 22 avoids this by permitting removal just through proximal retraction of the tether 22 out of the port 80.

While FIGS. 8A and 8B illustrate an endoscopic-based procedure, it should be understood that the flexible connector 10 may also be used in so called open-chest procedures wherein the patient undergoes a full sternotomy or large thoracotomy. In this embodiment, the insertion tool 30 may or may not be used as the surgeon may manually be able to advance (or retract) the flexible connector along the length of the ablation probe 50.

While the FLEX 10® ablation probe 50 described herein uses a moveable microwave antenna for ablation is should be understood that other ablation elements may also be utilized in connection with the ablation probe 50. For example, the ablation elements may include electrode(s) that are disposed along a length of the ablation probe 50. Still other ablation modalities known to those skilled in the art may be incorporated into the ablation probe 50.

While embodiments of the present invention have been shown and described, various modifications may be made without departing from the scope of the present invention. The invention, therefore, should not be limited, except to the following claims, and their equivalents.

What is claimed is:

1. A flexible connector for securing an ablation probe in a looped configuration comprising:
a flexible base member having first and second apertures dimensioned to receive the ablation probe and wherein the flexible base member is slidable along a length of the ablation probe when the ablation probe is positioned within the first and second apertures, the flexible base member further comprising a third aperture dimensioned to receive a looped portion of the ablation probe; and
a tether secured to the flexible base member.

2. The flexible connector of claim 1, wherein the flexible base member comprises a substantially flat base when not disposed on the ablation probe.

3. The flexible connector of claim 1, wherein the flexible base member comprises a tubular base when not disposed on the ablation probe.

4. The flexible connector of claim 1, wherein the tether is secured to a reinforcing member disposed in the flexible base member.

5. The flexible connector of claim 1, wherein the tether is secured to the flexible base member via one or more of the first, second, and third apertures.

6. The flexible connector of claim 1, wherein the tether comprises a suture.

7. The flexible connector of claim 1, wherein the flexible base member comprises a biocompatible elastomeric material.

8. The flexible connector of claim 1, wherein the apertures are circular.

9. The flexible connector of claim 1, wherein the apertures are polygonal.

10. The flexible connector of claim 1, further comprising a separate tubular member having a lumen therein for receiving the tether, the tubular member comprising an insertion tool for slidably moving the flexible connector along the length of the ablation probe.

11. An ablation probe comprising:
an elongate member having a proximal end and a distal end, the elongate member including an ablation antenna disposed at or near the distal end; and
a connector for securing a portion of the elongate member in a looped configuration, the connector comprising a base portion having an aperture dimensioned such that the base portion is slidable along a length of the elongate member, the connector including a tab projecting from the base portion and including an aperture therein, the aperture of the tab being oriented substantially perpendicular to the aperture of the base portion.

12. The ablation probe of claim 11, wherein the aperture in the base portion has a circular cross-sectional shape.

13. The ablation probe of claim 11, wherein the aperture in the base portion has a polygonal cross-sectional shape.

14. The ablation probe of claim 11, further comprising a tether secured to the connector.

15. The ablation probe of claim 11, wherein the tab comprises a biocompatible elastomeric material.

16. The ablation probe of claim 11, further comprising an insertion tool comprising an elongate tubular member having a lumen therein dimensioned for passage of a portion of the elongate member of the probe.

17. A method of positioning an ablation probe around the pulmonary veins of a patient using a flexible connector device having a tether attached thereto, comprising:
providing the flexible connector device on a proximal portion of the ablation probe;
routing the ablation probe around the pulmonary veins;
inserting a distal end of the ablation probe into an aperture contained in the flexible connector device; and
advancing the flexible connector device along a length of the ablation probe to form a looped portion around the pulmonary veins.

18. The method of claim 17, further comprising the step of withdrawing the flexible connector in a proximal direction by pulling on the tether.

19. The method of claim 17, wherein the flexible connector is advanced along the length of the ablation probe using an elongate pusher member having a lumen therein for receiving the tether.
